(19) United States
(12) Patent Application Publication
(10) Pub. No.: US 2008/0249533 A1
(43) Pub. Date: Oct. 9, 2008

(54) Medical Device and Method For Controlling Obesity

(75) Inventor: Norman Godin, Geneva (CH)

Correspondence Address: COZEN O'CONNOR, P.C., 1900 MARKET STREET, PHILADELPHIA, PA, 19103-3508, US

(73) Assignee: BIOMEDIX, S.A., Geneva (CH)

(21) Appl. No.: 12/066,025

(22) PCT Filed: Sep. 11, 2006
(86) PCT No.: PCT/US2006/035568

§ 371 Date: Mar. 6, 2008

Related U.S. Application Data
(60) Provisional application No. 60/715,442, filed on Sep. 9, 2005.
(60) Provisional application No. 60/747,709, filed on May 19, 2006.
(60) Provisional application No. 60/747,933, filed on May 22, 2006.

(51) Int. Cl. A61M 39/00; A61B 17/064

(52) U.S. Cl. 606/108; 606/219

(57) ABSTRACT

A method of, and device for, slowing the passage of food through a digestive tract of a patient and thereby treating obesity. The device is an obesity tube comprising (A) an upper ring of a size corresponding to a point under a patient's esophagus and above the patient's diaphragm muscle, and (B) a lower tube having a length and a distal opening. The method comprises stapling the upper ring under the patient's esophagus, above the patient's diaphragm muscle, and placing the lower tube distal to the upper ring. The length of the lower tube depends on whether the tube is to terminate distally in the stomach or terminate past the pylorus, in which case a section can be provided which is thick enough to resist collapsing under pylorus pressure. The lower tube can be entirely or partially non-permeable or semi-permeable. Semi-permeable tubes or sections thereof have walls which permit the passage of gastric hydrochloric acid but not food.

### CROSS-REFERENCE TO RELATED APPLICATIONS

Benefit of U.S. Provisional Application Ser. No. 60/715,442 filed Sep. 9, 2005, 60/747,709 filed May 19, 2006, and 60/747,933 filed May 19, 2006 are claimed, and the disclosures of said applications are hereby incorporated by reference.

### BACKGROUND OF THE INVENTION

The present invention relates to a medical prosthesis and method to help patients lose weight.

Excess weight and obesity have become a major health problem in developed nations. Medical authorities define obese as a body mass index (BMI) of 30 or above and overweight as a BMI of 25 or higher.

This preliminary observation is important in the sense that obesity has become a major public health issue in developed countries. It is estimated that “Each year an estimated 300,000 U.S. adults die of obesity-related causes, and the direct cost of obesity and physical inactivity has been estimated at 9.4% of U.S. health care expenditures,” according to Vincenza Snow, M D, et al., from the American College of Physicians, Philadelphia, Pa.

Diseases known to be associated with obesity include diabetes, hypertension, heart disease, sleep apnea, osteoarthritis of weight bearing joints, gallstones, infertility, increased incidence of carcinoma of the breast and prostate, and hiatus hernia with gastro-eophageal reflux disease known as GERD.

Obesity can be treated by diet and medication, however several drugs have been removed from the market because of side-effects such as fenfluramine, dexfenfluramine, and phenylpropanolamine. The drugs presently sold for obesity are sibutramine and orlistat, for example. According to a recent review published by Charles Vega in the April 2005 issue of the Annals of Internal Medicine, patients lose only 11 lbs on average after 6 months on these drugs.

The most common surgical treatment for obesity is Bariatric Surgery.

There are three main categories of bariatric surgery:
1) Gastric restrictive:
   A. Vertical Banded Gastroplasty (VBG) where a vertical line of staples are placed in the stomach creating a small pouch that empties in the larger stomach.
   B. Laparoscopic adjustable silicone gastric banding (LAP-BAND) has been very popular the last few years with surgeons and patients alike. The band is wrapped around the upper part of the stomach creating a small pouch of between 15 and 30 cc. In a study in 50 patients by Greenstein RJ, et al., in Obes. Surg. 1998 April; 8(2):199-206 entitled Esophageal anatomy and function in laparoscopic gastric restrictive bariatric surgery: implications for patient selection, the authors' conclusion was that patients with hiatus hernias and esophageal dysmotility were poor candidates for LAP-BAND because of a high rate of slippage of the band in hiatus hernia patients.
2) Malabsorptive procedures.
3) A combination of restrictive and malabsorptive procedures such as the Roux-en-Y gastric bypass are for more severely obese patients with BMIs of over 40. U.S. Patent publication 2004/0082963, Gannoe, et al, describes a device for use in tissue approximation and fixation which acquires tissue folds in a circumferential configuration within a stomach, creating a pouch or partition below the esophagus, and fastening the tissue folds such that a tissue ring or stomas forms, excluding the pouch from the greater stomach cavity. An optional bypass conduit from the tissue ring into the pylorus is also described. U.S. Pat. Pub. 2005/0075622 describes a tube which starts at the pylorus and has a metal reinforcement to avoid collapse at the pylorus.

Obesity is often associated with a hiatus hernia and GERD. Obese patients with a hiatus hernia do not all have GERD, and obese patients with GERD do not necessarily have a hiatus hernia. A normal lower esophageal pressure explains the absence of GERD in obese patients with a hiatus hernia.

Various treatments of treatment of GERD are known and used, for example, endoscopic gastroplasty, also called the Endocinch, which involves stitching pleats into the lower esophageal sphincter (LES) the muscle that regulates the flow of food from the esophagus to the stomach to reduce the backflow of acid.

Another endoscopic technique for treatment of GERD, the Stretta procedure, uses radio frequency to generate burning heat to the tip of a needle-like instrument. The heat is applied to the LES. The resulting scar tissue stiffens the sphincter and makes the sphincter more resistant to opening.

Another GERD treatment repairs the lower esophagus with an endoscope, using a gel called Enterix which reinforces the area. In an other approach, called the Gatekeeper, small prostheses are placed in the esophagus and expand to create a barrier to reflux. The Plicator—only recently approved by the FDA—is a device that is passed through the mouth into the stomach, where it places a suture that attempts to restore the anti-reflux barrier.

None of the prior endoscopic methods to treat GERD have been described as helping patients lose weight. All of these other methods are used in patients who have either no hiatus hernias or small hiatus hernias less than 2 cm. and these methods tend to reinforce or complement the weakened LES participating in GERD pathophysioloy.

In my previous patents: U.S. Pat. No. 6,764,518, Prosthesis for controlling the direction of flow in a duct of a living organism; U.S. Pat. No. 5,861,036, Medical prosthesis for preventing gastric reflux in the esophagus; and U.S. Pat. No. 5,314,473, Prosthesis for preventing gastric reflux into the esophagus, which are hereby incorporated by reference, I have described gastro-intestinal anti-reflux devices and methods of sizing and placing them endoscopically, which act as a substitute valve, prolonging artificially the esophagus in the stomach with thin collapsible walls at reflux pressures and which are for use in patients with more severe GERD associated with hiatus hernias. Hiatus hernias are known to be significantly aggravating factors for GERD.

### SUMMARY OF THE INVENTION

According to the present invention, thin-walled, tubes are implanted at or near the gastroesophageal junction (GES) of an overweight person and function to slow down passage of food so that the person must eat more slowly and chew their food more thoroughly than would otherwise be the case, inducing increased satiety. In certain embodiments, the tubes terminate in the stomach and do not pass the pylorus. In those embodiments preferably the tube is non-permeable. In certain other embodiments, where the tube is longer and designed to extend beyond the pylorus, either the proximal portion or the entire tube is semi-permeable such that it will allow gastric hydrochloric acid to pass in the tubes, which helps the breakdown of food in the tube and thereby helps food progress. When a portion or all of the tube is semi-permeable, gastric hydrochloric acid can penetrate the semi-permeable section of tube but the food content cannot exit through the wall of the tube.

In certain other embodiments, the tube is longer and extends past the pylorus, into the duodenum and jejunum, in which case preferably only the proximal gastric portion of the tube is semi-permeable. The portion of the tube that passes the pylorus has a thicker wall to avoid collapse through pyloric pressure when the pylorus contracts. The portion of the such tube in the duodenum is either semi-permeable or non permeable.

The tubes are placed through the mouth and can be retrieved through the mouth. In some embodiments an upper ring that is placed in a hernia after calibration with a calibration basket as described in my pending patent application PCT/US06/01181, which is hereby incorporated by reference. As disclosed therein, a catheter tube which is adapted to pass through the working channel of a endoscope or gastroscope that can be used under visual control to measure the diameter of a hollow organ such as the esophagus or hiatus hernia. In other embodiments, the ring of the obesity tube device can be placed in the lower esophagus.

The opening in conventional adult gastroscopes is usually 2.8 mm, but can vary between 2.0 mm and 5.0 mm for non-conventional gastroscopes such as pediatric gastroscopes or therapeutic endoscopes with larger channels. A video gastroscope can be used to assist in visualizing the measurement process with devices of the invention.

The catheter tube is placed through the working channel of the gastroscope until the last few inches or centimeters are visible. The lower esophagus or hiatus hernia are insufflated and the calibration basket is opened by pulling on the handle. The calibration basket is opened until the loops touch the mucosa of the hernia or wall of the organ measured on each side. The diameter of the opening is then read on the handle or the handle is opened up to a graduation that is read.

In another aspect, the invention comprises a method of slowing the passage of food through a digestive tract of a patient comprising stapling the upper ring of an obesity tube device, the device having (A) an upper ring and (B) a lower tube having a length and a distal opening, under the patient's esophagus, above the patient's diaphragm muscle, and placing the lower tube distal to the upper ring, the lower tube having at least one section made of material which is permeable to gastric hydrochloric acid but impermeable to solid food.

Preferably the upper ring is stapled to a hiatus hernia immediately under the patient's esophagus using either removable staples or transmural staples. If it becomes desired or necessary to remove the device, the staples can be removed or cut and the device removed through the mouth endoscopically. The device may be provided in several sizes with respect to the ring and with respect to the length of the tube. The ring size can be calibrated to the size of a particular patient's esophagus with a calibration basket and then a ring of an appropriate size to fit the location is selected and provided.

The tube is preferably single walled and straight, adapted to hang freely in the patient's stomach. While the section within the stomach is semi-permeable, any section passing within the pylorus is preferably formed from material which is thicker than the material of the first section. Any second section distal to the first section should be of a length to pass the patient's pylorus and of a thickness to avoid collapse through pyloric pressure, the first and second sections joined together so that food can pass continuously from the upper ring through the lower tube and out the distal lower opening. The overall length of the obesity tube, preferably about 10 to about 100 cm, is longer than that of my prior prosthesis disclosed in my above-referenced patents, and the thickness of the walls of the obesity tube is preferably about 1 to about 3 mm, and in some cases thicker, whereas the walls my aforementioned prior prosthesis tube are preferably about 0.5 mm thick.

If the device is intended to extend into the patient's duodenum or past that into the intestine, a third section distal to the second section is placed in the duodenum. The third section can be either permeable to gastric acid or non-permeable to gastric acid. Every section of the tube is joined together so that food can pass continuously from the upper ring through the lower tube and out the distal lower opening.

Optionally botulinum toxin is injected to reduce the strength of the patient's pyloric sphincter.

The device can be placed through the patient's mouth using an overtube placed in the esophagus, by inserting the obesity tube device in a placement tube while the overtube is in the esophagus, pushing the obesity tube distally with a forceps to force the obesity tube to eject from the placement tube and overtube, removing the placement tube, adjusting, if necessary, so that the ring is under the patient's esophagus, stapling the ring preferably with double tilt-tag staples as described in my above-referenced prior patent application, and placing the distal end of the lower tube in either the patient's stomach cavity or past the pylorus, depending on the selected length of the obesity tube and the desired distal location for a particular patient situation using an endoscope placed in the obesity tube with an endoscopy forceps placed through the working channel of the endoscope grabbing the end of the obesity tube and pushing it in place, and finally removing the overtube.

Certain prior art devices comprise a large annular element at the top that creates a reservoir at the top of the stomach. On the contrary, the tube of the present invention is placed immediately under the esophagus, in a hiatus hernia with no space for a reservoir. The ring of the invention is much narrower than such prior devices and is placed above the diaphragm muscle and not in the stomach per se. The devices of the invention do not have a funnel like cone in the top aspect and do not have a valve that opens and closes at the top level and at the pylorus. Further, the devices of the invention do not have a double-walled tube, with an interior aspect and an exterior aspect. The distal end of the obesity tube in the longer versions can be stapled in place to avoid displacement.

In the embodiments of the present invention where the tube extends into the pylorus, rather than metal reinforcement, preferably a thicker wall at the level of the pylorus is provided to avoid collapse.

In some embodiments, botulinum toxin may be injected to reduce the strength of the pyloric sphincter, as described by Friedenberg, et al, Dig Dis Sci. 2004 February; 49(2):165-75, where botulinum toxin was used for the treatment of gastrointestinal motility disorders.

Although the GARD is designed to treat GERD, as now described in previous my applications and patents, I have discovered with certain modifications and in certain embodiments a similar device acts as a kind of regulator of food intake by reducing the speed of food and in some cases the volume of food passing from the esophagus into the stomach.

The device and method of the invention enable decreasing the size of the reservoir of the stomach, slowing down the progression of food and, in some embodiments, blocking absorption. In the embodiments in which the tube extends past or into the duodenum, peristaltic contractions of the antrum, duodenum, and jejunum through a thin wall of the tube assist in food bolus progression.

### BRIEF DESCRIPTION OF THE DRAWINGS

FIG. 1 is a view of a gastrointestinal tract, partially in section, with a perspective view of a first embodiment of a device of the invention with the proximal end stapled to a hiatus hernia below the esophagus and above the diaphragm muscle, with the distal portion of the tube hanging freely in the stomach cavity.

FIG. 2 is a view of the gastrointestinal tract shown in FIG. 1, with a second, longer embodiment of a device of the invention, with the proximal end stapled to a hiatus hernia below the esophagus and above the diaphragm muscle, with the distal portion of the tube hanging freely in the stomach cavity.

FIG. 3 is a view of the gastrointestinal tract shown in FIGS. 1 and 2, with a third, still longer embodiment of a device of the invention, with the proximal end stapled to a hiatus hernia below the esophagus and above the diaphragm muscle, with the distal portion of the tube passing the patient's pylorus and located in the duodenum.

FIG. 4 is a view of the gastrointestinal tract shown in FIGS. 1-3, with a fourth, still longer embodiment of a device of the invention, with the proximal end stapled to a hiatus hernia below the esophagus and above the diaphragm muscle, with the distal portion of the tube located in the patient's fourth portion of the duodenum.

FIG. 5 is a view of the gastrointestinal tract shown in FIGS. 1-4, with a fifth, still longer embodiment of a device of the invention, with the proximal end stapled to a hiatus hernia below the esophagus and above the diaphragm muscle, with the distal portion of the tube located in at the junction of the patient's duodenum and jejunum.

FIG. 6 is a view of the gastrointestinal tract shown in FIGS. 1-5, with a sixth, still longer embodiment of a device of the invention, with the proximal end stapled to a hiatus hernia below the esophagus and above the diaphragm muscle, with the distal portion of the tube located past the patient's duodenum and partially within the patient's intestine (jejunum).

FIGS. 7a, 7b and 7c are three sequential side views, partially in cross-section, showing the obesity tube being pulled into a placement tube with forceps.

FIGS. 8a, 8b and 8c are three sequential views of the placement tube being inserted into an overtube which has first been placed in an esophagus and then pushing the obesity tube out with forceps.

### DETAILED DESCRIPTION

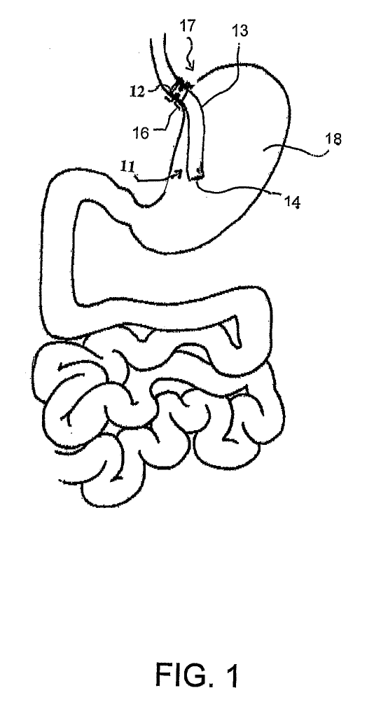

Referring first to FIG. 1, a device 11 having (A) an upper ring 12 and (B) a lower tube 13 having a length and a distal opening 14 is fixed with staples 16 at a point 17 under the patient's esophagus and above the patient's diaphragm muscle, the lower tube 13 hanging freely in the stomach cavity 18. The tube 13 is either completely impermeable to hydrochloric acid and other gastric fluids as well as impermeable to food or has at least one section made of material which is permeable to gastric hydrochloric acid but impermeable to solid food, referred to sometimes herein as “semi-permeable.” In the embodiment shown in FIG. 1, the entire distal portion of the tube is constructed of the same non permeable medical grade biocompatible synthetic polymer. The preferred polymers are silicone, polyurethane, polyester, and polytetrafluoroethylene (PTFE). The staples 12 can be transmural and thus non-removable, or can be of the wing type which are removable.

The obesity tube device 11 can be placed endoscopically through conventional overtubes such as the “Guardus” overtube system of U.S. Endoscopy, presently available under product codes 00711146, 00711147, 00711148, or 00711149, and the ring 12 can be stapled to a hiatus hernia or other location below the esophagus and above the diaphragm muscle using endoscopic stapling techniques described in our patent application Ser. No. 11/215,904 or PCT/US05/30725 through Guardus or similar overtube systems.

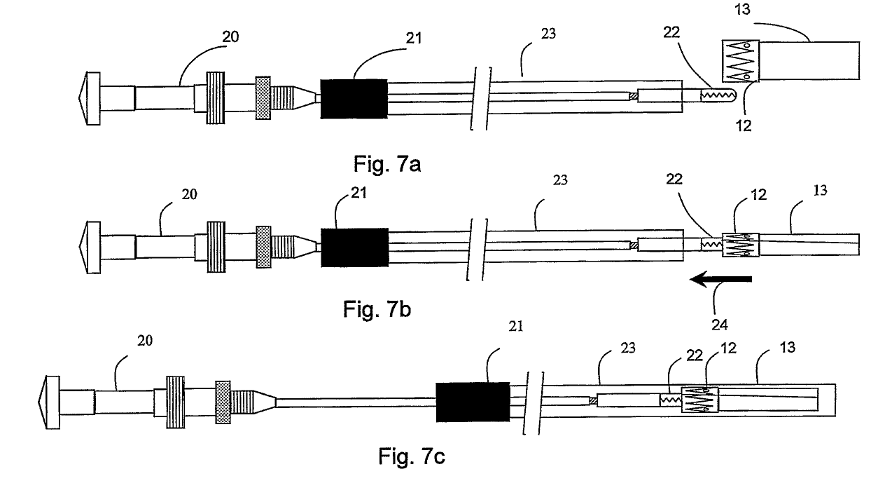

Referring to FIG. 7a, a forceps having handle 20 and distal operating end 22 is shown in placement tube 21 which has distal portion 23. The obesity tube having ring 12 and tube 13 is shown uncompressed in FIG. 7a, and then compressed in FIG. 7b, being held by the forceps distal end 22 and pulled in the direction of arrow 24 into the placement tube. FIG. 7c shows the obesity tube device 12, 13 having been pulled into the placement tube.

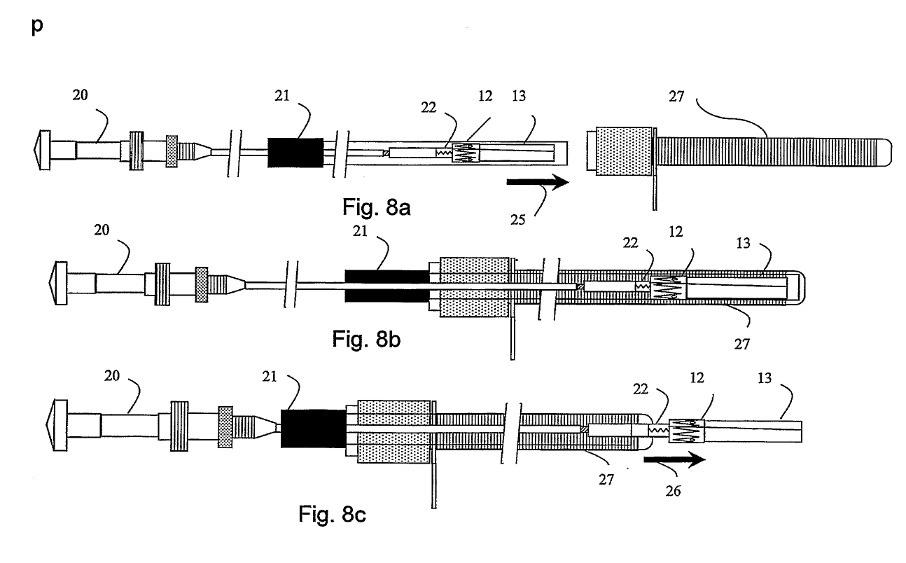

Referring to FIG. 8a, the placement tube 21, now containing the forceps 22 and obesity tube device 12, 13 can be inserted in the direction of arrow 25 into the overtube 27 which has just previously been inserted through the mouth and esophagus. Once the placement tube 21 is in the overtube 27, as shown in FIG. 8b, the handle 20 of the forceps 22 can be used to push the obesity tube device forward in the direction of arrow 26 in FIG. 8c, to force the obesity tube to eject from the placement tube and overtube. Then the forceps and placement tube are removed after adjusting the location of the ring 12, if necessary, so that the ring is under the patient's esophagus. The ring can be stapled through the overtube, and placing the distal end of the lower tube either falls naturally into the stomach cavity or is guided there or through the pylorus with endoscope and small forceps passed through the working channel of the endoscope, depending on the selected length of the obesity tube and the desired distal location for a particular patient situation. When the stapling and placing of the obesity tube are complete, the overtube is removed. The distal end in the longer versions reaching the duodenum can be stapled to avoid displacement.

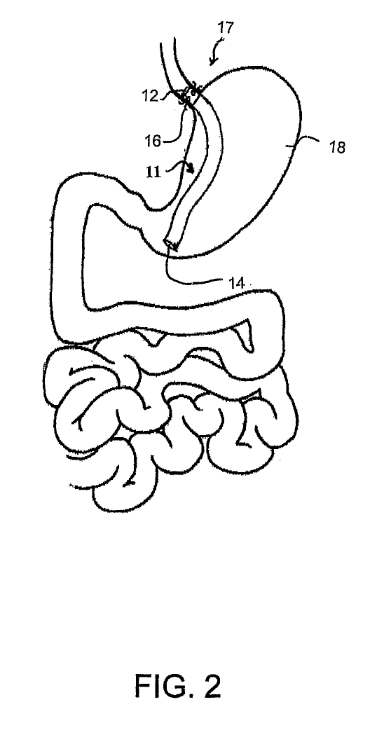

FIG. 2 illustrate a second embodiment of the obesity tube which is longer than that of FIG. 1 and hangs further into the stomach cavity, but is otherwise the same in function and construction.

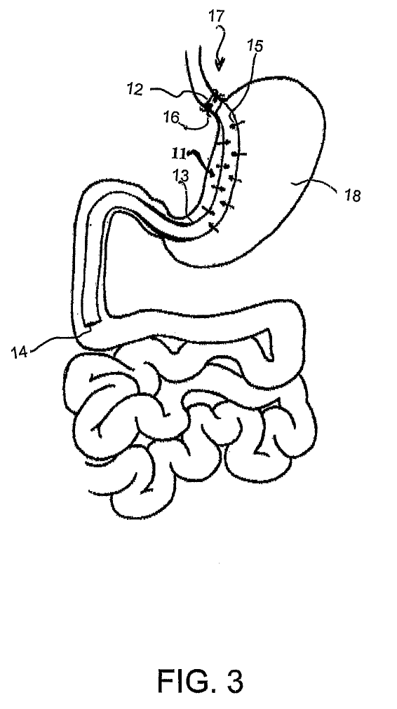

FIG. 3 illustrates a third, still longer embodiment of a device of the invention, with the proximal end stapled to a hiatus hernia below the esophagus and above the diaphragm muscle, with the distal portion of the tube located in the patient's duodenum. The section of the tube within the pylorus is thicker, preferably between 1 and 3 mm in thickness and 10 mm and 30 mm in diameter, preferably about 20 mm to 30 mm in diameter and 3 cm. to 10 cm. in length. Arrows 15 pointing in toward the tube 13 within the stomach cavity illustrate passage of gastric hydrochloric acid, which is optional.

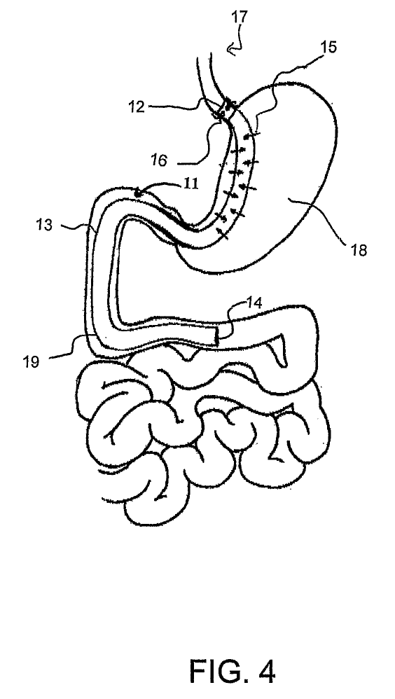

FIG. 4 is a view of a fourth embodiment of device 11 with a longer tube 13 which passes the pylorus and terminates in the duodenum. In this embodiment, there is a total gastric bypass and a partial duodenum bypass. The portion of the tube 19 in the duodenum in this embodiment is semi-permeable, but the portion 13 passing through the pylorus is not permeable since it is constructed of a thicker material so that it is resistant to pyloric pressure and thereby avoids collapse.

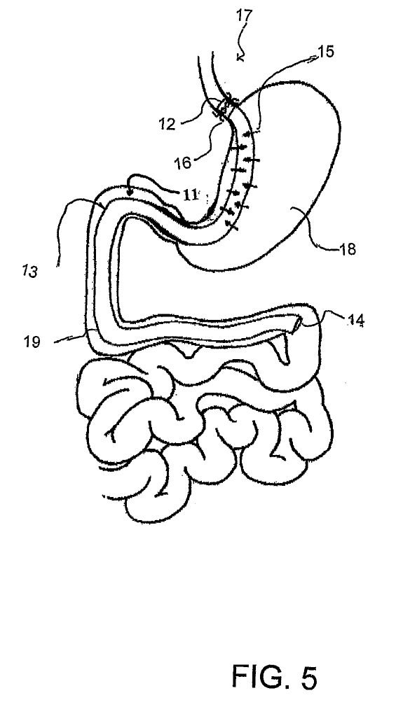

FIG. 5 is a view of a fifth embodiment of device 11 with a longer tube 13 which passes the pylorus and extends throughout the duodenum. In this embodiment, there is a total gastric and duodenum bypass. The portion of the tube 13 in the duodenum in this embodiment is, non permeable as well as the portion passing through the pylorus is not permeable since it is constructed of a thicker material so that it is resistant to pyloric pressure and thereby avoids collapse. The proximal part in the stomach in this embodiment can be either non-permeable or semi-permeable.

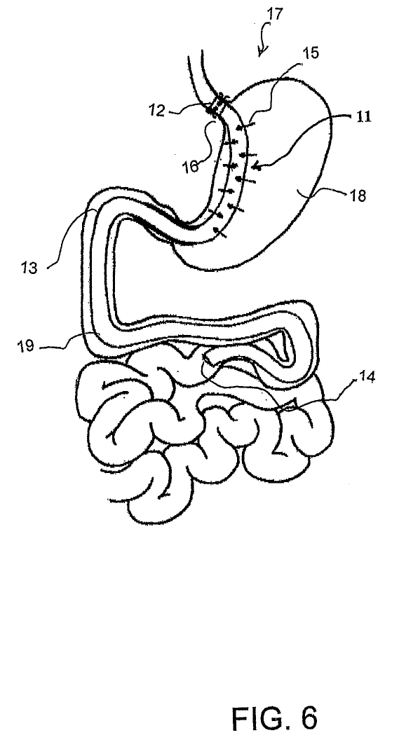

FIG. 6 is a view of a sixth embodiment of device 11 with a longer tube 13 which passes the pylorus and extends past the duodenum and partially into the small intestine. In this embodiment, there is a total gastric and duodenum bypass. The portion of the tube 13 in the duodenum in this embodiment is non permeable, as well as the portion passing through the pylorus that is not permeable since it is constructed of a thicker material so that it is resistant to pyloric pressure and thereby avoids collapse.

An advantage of the method of the invention is that the obesity device 11 is placed through the mouth without surgery. The device 11 diameter and volume capacity can be calibrated so as to allow volumes of acceptable meals for the patient and the outflow of food from the device into the stomach, pylorus, duodenum, or intestine (jejunum) is controlled. stapled

### EXAMPLES

The following non-limiting example is presented to illustrate one embodiment of the invention. In this example, the anti-reflux device for the treatment of GERD, described in U.S. Pat. No. 5,861,036, held in place with a ring as described in U.S. Pat. No. 6,764,518 in an obese patient with GERD and a hiatus hernia who could not lose weight on a conventional therapy of diet and exercise. It was observed with great surprise that the patient lost a significant amount of weight.

The device had a volume of approximately 50 cc. This 61 year old male subject of this example had had a failed open Nissen fundoplicature operation for GERD 15 years previously and had severe pathological reflux as measured by 24 hour pH metric testing in the esophagus while on medical therapy, that is double dose proton pump inhibitors (Pantoprazole 40 mg BID). He refused repeat surgery.

The subject patient accepted to enter a preliminary trial a tubular valve of the invention for a period of 6 months. The tubular valve and ring was placed through the mouth in the patient's hiatus hernia with the tubular valve at the cardia. The patient was placed on a liquid diet for 2 days after positioning the device, then asked to resume his normal diet while avoiding spicy foods and alcohol. All medications were withdrawn.

As expected, one month later, there was no reflux at all at repeat pH metric testing despite the very severe reflux that the patient had had before the implantation of the device.

However, the unexpected and surprising observation was that the patient lost about 10 kg (about 22 pounds) in the few months following implantation without a particular diet. He had to eat slowly, only swallowing smaller pieces of food than previously to allow passage of food through the tubular device acting as a kind of funnel or reservoir. This patient's BMI decreased from 32.4 to 29.3 in 6 months. This helped the patient pass from being obese to overweight only (BMI under 30).

While the invention has been described in detail and several embodiments have been illustrated, other embodiments, alternatives, and modifications should become apparent to those skilled in the art without departing from the spirit and scope of the invention.

What is claimed is:

1. A method of slowing the passage of food through a digestive tract of a patient comprising stapling the upper ring of an obesity tube device, the device having (A) an upper ring and (B) a lower tube having a length and a distal opening, under the patient's esophagus, above the patient's diaphragm muscle, and placing the lower tube distal to the upper ring.

2. The method of claim 1, the lower tube is completely impermeable to gastric hydrochloric acid and to solid food.

3. The method of claim 1, the lower tube having at least one section made of material which is permeable to gastric hydrochloric acid but impermeable to solid food.

4. The method of claim 1 comprising stapling the upper ring to a hiatus hernia immediately under the patient's esophagus using either removable staples or transmural staples.

5. The method of claim 1 comprising calibrating the size of the location under the patient's esophagus with a calibration basket and providing a ring of an appropriate size to fit the location.

6. The method of claim 1 wherein the tube is single walled and straight, adapted to hang freely in the patient's stomach.

7. The method of claim 1, wherein the obesity tube device has a second section comprised of material which is thicker than the material of the first section, the second section distal to the first section and of a length to pass the patient's pylorus and of a thickness to avoid collapse through pyloric pressure, the first and second sections joined together so that food can pass continuously from the upper ring through the lower tube and out the distal lower opening.

8. The method of claim 1 wherein the obesity tube device has a second section comprised of material which is thicker than the material of the first section, the second section distal to the first section and of a length to pass the patient's pylorus and of a thickness to avoid collapse through pyloric pressure, and a third section distal to the second section and placed in the patient's duodenum, the third section being either permeable to gastric acid or non-permeable to gastric acid, the sections joined together so that food can pass continuously from the upper ring through the lower tube and out the distal lower opening.

9. The method of claim 1 wherein the obesity tube device has a second section comprised of material which is thicker than the material of the first section, the second section distal to the first section and of a length to pass the patient's pylorus and of a thickness to avoid collapse through pyloric pressure, the first and second sections joined together so that food can pass continuously from the upper ring through the lower tube and out the distal lower opening, comprising the step of injecting botulinum toxin to reduce the strength of the patient's pyloric sphincter.

10. The method of claim 1 comprising placing the obesity tube device in a placement tube, placing an overtube through the patient's mouth into the patient's esophagus, placing the placement tube containing the obesity tube device in the overtube while the overtube is in the esophagus, pushing the obesity tube distally with a forceps to expel the obesity tube device from the placement tube and the overtube, stapling the ring under the patient's esophagus above the diaphragm muscle, placing the distal end of the lower tube in the patient's stomach cavity, passing a long endoscope in the tube in order to grab the distal end with a forceps and place the distal end in the duodenum and/or jejunum, and removing the placement tube and the overtube.

11. The method of claim 1 comprising placing the obesity tube device in a placement tube, placing an overtube through the patient's mouth into the patient's esophagus, placing the placement tube containing the obesity tube device in the overtube while the overtube is in the esophagus, pushing the obesity tube distally with a forceps to expel the obesity tube device from the placement tube and the overtube, stapling the ring under the patient's esophagus above the diaphragm muscle, and placing the distal end of the lower tube past the patient's pylorus with an endoscope so that a section of the lower tube distal to the first section having a thickness sufficient to avoid collapse through pyloric pressure is placed within the patient's duodenum, with the distal end stapled in place.

12. The method of claim 10 comprising placing a third section of the tube of the obesity device in the patient's duodenum, with the distal end hanging loose.

13. An obesity tube device comprising (A) an upper ring of a size corresponding to a point under a patient's esophagus and above the patient's diaphragm muscle, and (B) a lower tube having a length and a distal opening leave in, the device adapted to slow the passage of food through the patient's digestive tract.

14. The device of claim 13, the lower tube having being completely non permeable to gastric fluids and to foods.

15. The device of claim 13, the lower tube having at least one section made of material which is permeable to gastric hydrochloric acid but impermeable to solid food.

16. The obesity tube device of claim 13 of a length adapted to hang freely in the patient's stomach when the ring is stapled to the point under the patient's esophagus and above the patient's diaphragm muscle.

17. The obesity tube device of claim 13 of a length adapted to extend pass the patient's pylorus when the ring is stapled to the point under the patient's esophagus and above the patient's diaphragm muscle, the tube having a first section which is permeable to gastric hydrochloric acid and impermeable to the passage of food, and a second section distal to the first section which is of a thickness sufficient to avoid collapse through pyloric pressure, the sections joined together so that food can pass continuously from the upper ring to the distal lower opening.

18. The obesity tube device of claim 13 of a length adapted to extend pass the patient's pylorus when the ring is stapled to the point under the patient's esophagus and above the patient's diaphragm muscle, the tube having a first section which is permeable to gastric hydrochloric acid and impermeable to the passage of food, and a second section distal to the first section which is of a thickness sufficient to avoid collapse through pyloric pressure, and a third section distal to the second section, the third section adapted for placement in the patient's duodenum so that the distal opening of the tube is within the duodenum, the sections joined together so that food can pass continuously from the upper ring to the distal lower opening.

19. The obesity tube device of claim 13 wherein the one or more sections of the tube are constructed of biocompatible medical grade polyester, polytetrafluoro ethylene (PTFE), silicone, and/or polyurethane.
